(19) United States
(12) Patent Application Publication
(10) Pub. No.: US 2024/0277517 A1
(43) Pub. Date: Aug. 22, 2024

(54) DRUG DELIVERY DEVICE WITH CONJUNCTIVAL COMPRESSION PAD

(71) Applicant: Twenty Twenty Therapeutics LLC, South San Francisco, CA (US)

(72) Inventors: Alexandre R. Tumlinson, South San Francisco, CA (US); Dimitri Azar, South San Francisco, CA (US); Shannon Kozinn, South San Francisco, CA (US)

(21) Appl. No.: 18/112,019

(22) Filed: Feb. 21, 2023

(51) Int. Cl. A61F 9/00; A61B 10/00

(52) U.S. Cl. CPC A61F 9/0017; A61B 10/0045; A61B 2017/12004

(57) ABSTRACT

An intravitreal injection device is provided that includes a therapeutic agent reservoir and a hollow eye penetration member sized and dimensioned to be inserted into the vitreous humor of a patient's eye and in fluid communication with the therapeutic agent reservoir. A tamponade pad configured to contact and apply pressure to a conjunctiva of the patient's eye is translationally coupled to the hollow eye penetration member. The translational coupling between the tamponade pad and the hollow eye penetration member allows the withdrawal of the hollow eye penetration member while maintaining pressure between the tamponade pad and the conjunctiva.

### TECHNICAL FIELD

The present disclosure relates to an intravitreal injection device with a compression pad configured to minimize conjunctival hemorrhaging.

### BACKGROUND

Vitreous humor is a colorless, gelatinous fluid within an eye or eyeball of humans or other vertebrates composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites and nutrients. It is produced largely by cells of the ciliary body. Changes in vitreous structure that occur with aging, are important in the pathogenesis of many vitreoretinal diseases.

Intraocular pressure (IOP) quantifies the pressure of the vitreous humor inside the eye. Many individuals suffer from disorders, such as glaucoma, that are associated with chronic heightened IOP. Over time, heightened IOP can cause damage to the optical nerve of the eye, leading to loss of vision.

Presently, treatment of ophthalmic disorders mainly involves periodically administering pharmaceutical agents to the eye. These drugs can be delivered by, for example, intravitreal injection (“IVI”). Intravitreal injection is one of the most common surgical procedures performed in ophthalmology today. A variety of drugs are delivered directly to the clear vitreous gel that supports the globe of the eye. These drugs act directly in the vitreous or in the surrounding retinal tissues over the following months. For example, intravitreal injection is a common route of delivery for vascular endothelial growth factor inhibiting (anti-VEGF) proteins, which are highly potent compounds tolerated at high doses, with intravitreal half-lives about one weck. Anti-VEGF biologics and steroids are the most commonly administered drugs by this route. These drugs may be administered on a chronic basis.

One recommended procedure for intravitreal injection includes preparation of an injection needle, topical anesthesia and disinfection of the eye surface, holding the eye open with a lid speculum or other means, optional lateral dislocation of the conjunctiva at the injection site, and insertion of the needle a few mm lateral to the limbus to approximately the full depth of the needle, injecting the drug, withdrawing the needle, and allowing the conjunctiva to cover the injection site. Post injection care typically includes a basic verification of functional vision such as requesting the patient to count the number of fingers shown by the doctor. This functional test verifies that acute IOP increase due to injection has not impacted the optic nerve head in a way that requires immediate relief.

Another important ophthalmic procedure is vitreous sampling. Vitreous sampling may inform various aspects of eye care. Samples of vitreous may be analyzed for cellular content and extracellular structure by histology or immunologic analysis. Histology can, for example, provide a definitive diagnosis for the type of infection causing endophthalmitis.

Identification of the type of immune cells present and the immune mediator proteins expressed may inform the treatment of uveitis. Identification of the amount of VEGF present in the vitreous may give an indication of how likely imminent neovascularization is to occur or how likely it is that VEGF compounds are responsible for an observed case of neovascularization. Non-responders to anti-VEGF treatment remains one of the most troublesome aspects of treating neovascularization in exudative, age-related vascular degeneration (also known as wet AMD) and diabetic retinopathy.

Two common methods of vitreous sampling—with a cutter or with needle aspiration—appear to be approximately equivalent for the purposes of protein analysis. A state of the art miniature cutting tool may be delivered through a 23-gauge trocar. Needle aspiration may be performed with needles as small as 30-gauge (about half the diameter of 23 gauge). Fine gauge may increase the probability of a dry tap and/or change the properties of the aspirated material by acting as a filter. Small gauge may have an advantage in that traction may not be introduced on the gel matrix because the gel matrix cannot be pulled into the small needle bore. Vitreous samples are typically frozen or otherwise stabilized so that they can be processed in a laboratory outside of the operating room or ophthalmic office setting.

Injection of therapeutic doses of medication into the vitreous or aqueous humor inside the eye can increase IOP by as much as 25 mmHg, which is substantially greater than threshold levels that are considered potentially harmful. Evidence shows that while such IOP increases are transient, they are in fact associated with an iatrogenic glaucoma resulting in measurable loss of nerve fiber layer and visual function over a course of only several treatments in patients with ‘normal’ resting IOP. See Saxena, S., Lai, T. Y., Koizumi, H. et al., “Anterior chamber paracentesis during intravitreal injections in observational trials: effectiveness and safety and effects,” International Journal of Retina and Vitreous, 5, 8 (2019). Therefore, it is sometimes desirable to remove a small volume of humor (whether aqueous, vitreous or both) from the eye before injecting a comparable volume of medication. However, removal of a volume of humor may result in insufficient pressure, which can also be harmful to the eye.

Therefore, in the case of diagnostic sampling of humors, it may be necessary or beneficial to inject a volume of fluid (whether medicated or otherwise) to replace the withdrawn humors. In either case, care must be taken to ensure that the removed and injected volumes are comparable, and in either case, two separate procedures (a sampling procedure and an injection procedure) are typically required.

Conjunctival hemorrhage is a common side effect of intravitreal injection where a superficial conjunctival blood vessel, broken by the needle continues to bleed after the needle is removed from the eye. The resulting bright red patch on the eyeball is visible for several days after the injection. Although primarily of cosmetic importance, the injury greatly affects patient satisfaction. Physicians may take extra time to apply pressure with a sterile cotton tipped swab for several seconds to a minute to the affected area to tamponade the bleeding.

### SUMMARY

The present disclosure relates to an intravitreal injection device with a conjunctival suppression pad. The intravitreal injection device can be used for drug delivery as well as aspiration. The conjunctival suppression pad can provide a contact surface to maintain pressure on the IVI injection site momentarily after completion of the IVI to suppress conjunctival bleeding. The contact surface may also be adapted to absorb refluxed vitreous fluid in order to acquire a sample for analysis.

In an aspect, an intravitreal injection device can comprise a therapeutic agent reservoir and a hollow eye penetration member sized and dimensioned to be inserted into the vitreous humor of a patient's eye and in fluid communication with the therapeutic agent reservoir. A tamponade pad can be configured to contact and apply pressure to a conjunctiva of the patient's eye and can be translationally coupled to the hollow eye penetration member. The translational coupling between the tamponade pad and the hollow eye penetration member can allow the withdrawal of the hollow eye penetration member while maintaining pressure between the tamponade pad and the conjunctiva.

### BRIEF DESCRIPTION OF THE DRAWINGS

FIG. 1 is a schematic illustration of an intravitreal injection device according to an aspect of the present disclosure.

FIG. 2 is a side sectional view of an intravitreal injection device according to an aspect of the present disclosure.

FIG. 3 is a side sectional view of an intravitreal injection device according to an aspect of the present disclosure.

FIG. 4 is a side sectional view of an intravitreal injection device according to an aspect of the present disclosure.

FIGS. 5A-5E depict the configuration of intravitreal injection device of FIG. 3 during different steps of deployment according to an aspect of the present disclosure.

FIGS. 6A-6E depict an alternative configuration of the intravitreal injection device of FIG. 3 during different steps of deployment according to an aspect of the present disclosure.

FIGS. 7A-7E depict the configuration of intravitreal injection device of FIG. 4 during different steps of deployment according to an aspect of the present disclosure.

FIG. 8 is a side sectional view of an intravitreal injection device according to an aspect of the present disclosure.

FIG. 9 is a side sectional view of an intravitreal injection device according to an aspect of the present disclosure.

FIGS. 10A-10E depict the configuration of the intravitreal injection device of FIG. 8 during different steps of deployment according to an aspect of the present disclosure.

FIG. 11 is a side sectional view of an intravitreal injection device according to an aspect of the present disclosure.

FIG. 12 is a side sectional view of an intravitreal injection device according to an aspect of the present disclosure.

FIGS. 13A-13E depict the configuration of the intravitreal injection device of FIG. 11 during different steps of deployment according to an aspect of the present disclosure.

FIG. 14 is a side sectional view of an intravitreal injection device according to an aspect of the present disclosure.

FIG. 15 is a side view of components of an intravitreal injection device according to an aspect of the present disclosure illustrating a sample of vitreous fluid or gel that has been absorbed by a tamponade pad from aspirated from the vitreous humor of a patient's eye and therapeutic agent that has been delivered in the patient's eye.

FIG. 16 a side view of components of an intravitreal injection device according to an aspect of the present disclosure illustrating a sample of vitreous fluid or gel that has been aspirated from the vitreous humor of a patient's eye and protected by a cover.

FIG. 17 illustrates a sample of vitreous fluid or gel absorbed by a tamponade pad of an intravitreal injection device that has been transferred to a container for further analysis.

FIG. 18 is a side sectional view of components of an intravitreal injection device according to an aspect of the present disclosure when placed against a patient's eye with a sample of vitreous fluid or gel that has been aspirated from the vitreous humor of a patient's eye and therapeutic agent that has been delivered in the patient's eye.

FIG. 19 is a side sectional view of the device components illustrated in FIG. 18.

FIG. 20 is a side sectional view of the components of the device illustrated in FIG. 18 where an aspirated sample has been placed in a solvent for further analysis.

FIG. 21 is a side sectional view of an intravitreal injection device according to an aspect of the present disclosure.

FIGS. 22A-22F depict the configuration of the intravitreal injection device of FIG. 21 during different steps of deployment according to an aspect of the present disclosure.

FIG. 23 is a side sectional view of an intravitreal injection device according to an aspect of the present disclosure.

FIG. 24 is a side sectional view of an intravitreal injection device according to an aspect of the present disclosure.

FIG. 25A is a side sectional view illustrating vitreous strands that may be attached to a sample of vitreous fluid or gel after aspiration.

FIG. 25B-25E depict the configuration of an intravitreal injection device during different steps of deployment according to an aspect of the present disclosure.

### DETAILED DESCRIPTION

The present disclosure relates to an IVI device with a tamponade pad. By “tamponade” pad is meant a pad that is configured, sized, and dimensioned to apply sufficient compressive force to the conjunctiva of a patient's eye to suppress, mitigate, or prevent conjunctival bleeding. As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” is meant that the distance, shape, or configuration of the described element need not have the mathematically exact described distance, shape, or configuration of the described element but can have a distance, shape, or configuration that is recognizable by one skilled in the art as generally or approximately having the described distance, shape, or configuration of the described element. As such “substantially” refers to the complete or nearly complete extent of a characteristic, property, state, or structure. The exact allowable degree of deviation from the characteristic, property, state, or structure will be so as to have the same overall result as if the absolute characteristic, property, state, or structure were obtained “connected to” “operably connected to,” “coupled to,” “operably coupled to,” “disposed adjacent to,” “disposed between,” “disposed on.” “between,” “located at” another component can have intervening components between the components so long as the device can perform the stated purpose. The sequence of operations (or steps) is not limited to the order presented in the claims or figures unless specifically indicated otherwise. As used herein a “patient” includes a mammal such as a human being. Although the drawings show certain elements of a device in combination, it should be noted that such elements can be included (or excluded) in the depicted embodiment and in other embodiments or aspects illustrated in other drawings. In other words, each of the disclosed aspects and embodiments of the present disclosure may be considered individually or in combination with other aspects and embodiments of the disclosure. All devices and components as described herein are used for medical purposes and are therefore sterile.

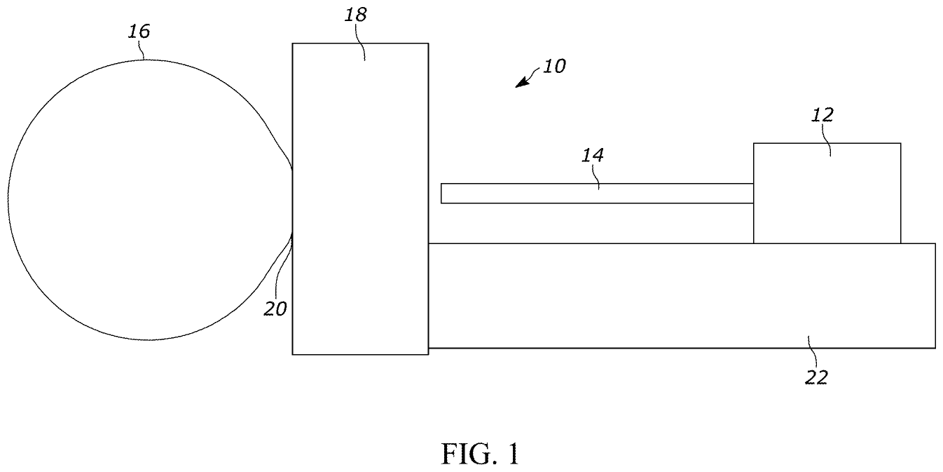

Referring to FIG. 1, in an aspect, intravitreal injection device 10 can comprise therapeutic agent reservoir 12 and hollow eye penetration member 14 sized and dimensioned to be inserted into the vitreous humor of a patient's eye 16 and in fluid communication with therapeutic agent reservoir 12. The therapeutic agent reservoir can be pre-filled or manually filled when needed. The hollow eye penetration member can be, for example, a needle, trocar, cannula or other surgical component that is configured to be inserted into the eye's vitreous humor. Device 10 can include tamponade pad 18 that is configured to contact and apply pressure to a conjunctiva 20 of the patient's eye 16 and can be translationally coupled to hollow eye penetration member 14. Such translational coupling between tamponade pad 18 and hollow eye penetration member 14 can allow the withdrawal of hollow eye penetration member 14 while maintaining pressure between tamponade pad 18 and conjunctiva 20. In particular, the tamponade pad is configured to transmit compressive forces to the conjunctiva and thereby compress the tissue (e.g. the conjunctiva) underneath the tamponade pad. The tamponade pad can be fabricated of a suitable material and sized and dimensioned to transmit compressive forces to the conjunctiva to minimize or prevent conjunctiva hemorrhaging. For example, the tamponade pad can be fabricated of an absorbent material (described in more detail below), a rigid or semi-rigid plastic, or other suitable material to transmit such compressive forces to the conjunctiva. To the extent the tamponade pad has a generally circular or oval shape, the tamponade pad can have a diameter between about 3.5 millimeters (mm) and about 4 mm. In certain aspects and as described in more detail below, the hollow eye penetration member can be self-retracting. For example, spring 22 can be located adjacent to hollow eye penetration member 14 and proximal to tamponade pad 18. Spring 22 can have a compressed state in which hollow eye penetration member 14 extends distally beyond tamponade pad 18 and an expanded state in which hollow eye penetration member 14 extends proximal to tamponade pad 18. Spring 22 is only one example of how to implement a self-retracting hollow eye penetration member. Other components could also be utilized such that the hollow eye penetration self-retracts while pressure is preferably still applied to the tamponade pad.

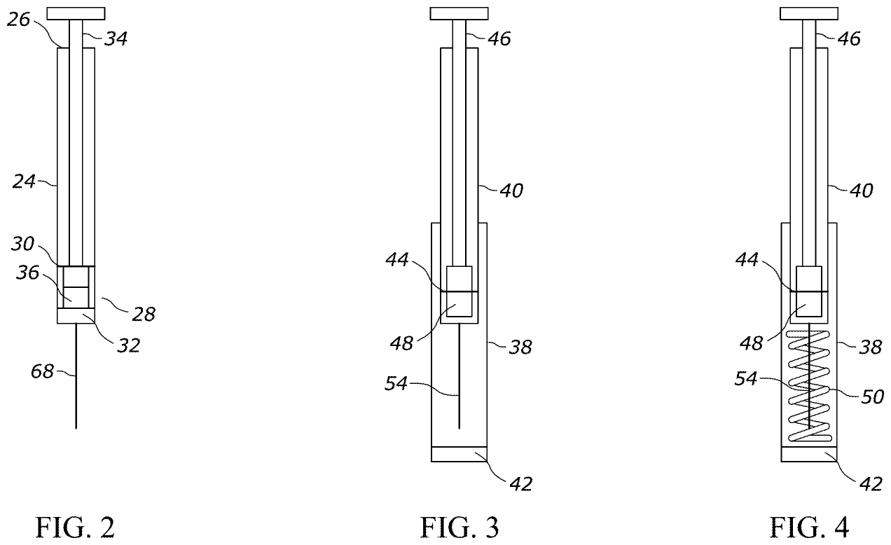

Referring to FIGS. 2-4, an intravitreal injection device can further comprise a single housing or concentric housings that contain or are in communication with the above-described components of an intravitreal injection device. For example and with reference to FIG. 2, an intravitreal injection device can include housing 24 having a proximal end 26 and a distal end 28 with tamponade pad 32 located at distal end 28 and therapeutic agent reservoir 30 contained within housing 24 proximal to tamponade pad 32. Plunger 34 can be located proximal to therapeutic agent reservoir 30 and longitudinally displaceable within housing 24 to urge therapeutic agent 36 contained in therapeutic agent reservoir 30 into the patient's eye. Alternatively and with reference to FIGS. 3 and 4, an intravitreal injection device can include

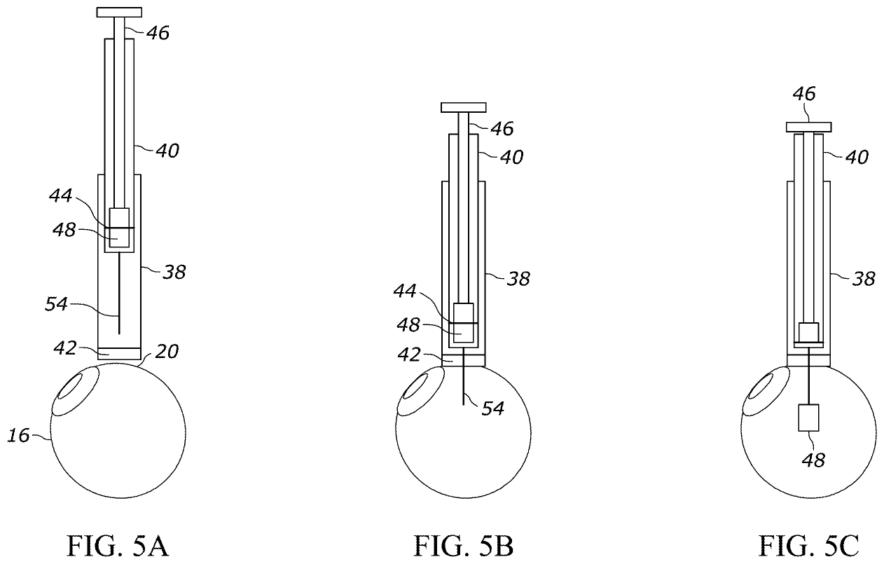

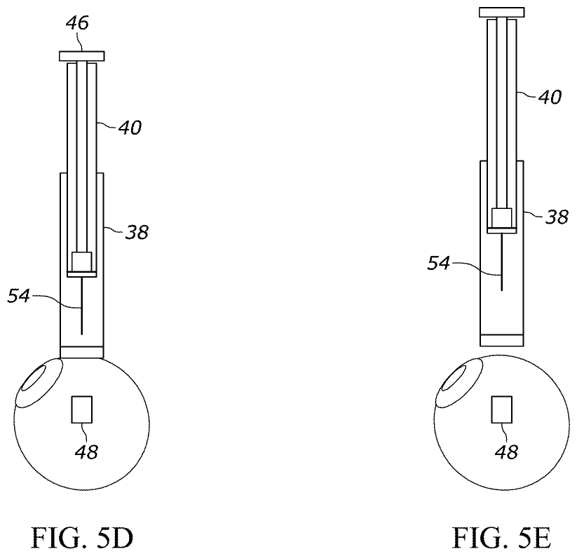

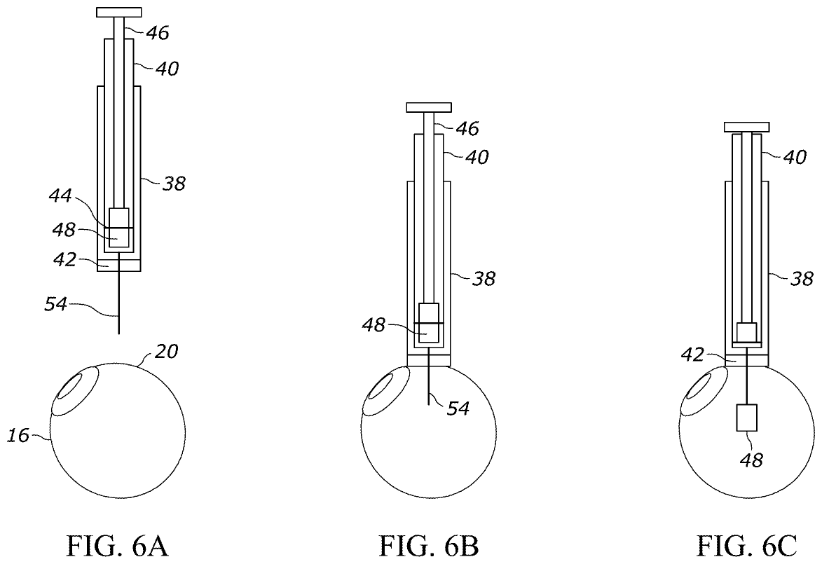

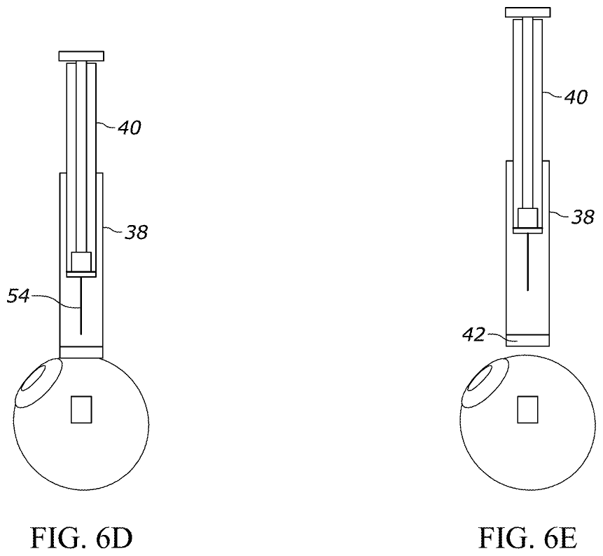

outer housing 38 and inner housing 40 slidably disposed in outer housing 40. Tamponade pad 42 can located at the distal end of outer housing 38 and therapeutic agent reservoir 44 can be located within the inner housing 40. Plunger 46 can be located proximal to therapeutic agent reservoir 44 and longitudinally displaceable within inner housing 40 to urge therapeutic agent 48 contained in therapeutic agent reservoir 44 into the patient's eye. FIGS. 5 and 6 illustrate the relative position of components of the intravitreal injection device depicted in FIG. 3 during different stages of deployment. In FIG. 5A, the intravitreal injection device is depicted with hollow eye penetration member disposed completely within outer housing 38 entirely proximal to tamponade pad 42. Such a configuration can allow a user to visualize the tamponade pad during deployment such that the pad effectively serves as a guide to assist the user in correctly aligning the device with the correct insertion site of the patient's eye. For example, the visualization of the pad could serve as a guide to the user to place the pad such that border of the pad is placed directly adjacent to the limbus of the patient's eye so that the hollow eye penetration member enters the pars plana approximately 3.5 millimeters (mm) to approximately 4 mm posterior to the limber. In FIG. 6A, the intravitreal injection is illustrated with hollow eye penetration member 54 partially extending through tamponade pad 42 such that the distal portion of hollow eye penetration member 54 is “exposed” at the beginning of the procedure. Such a configuration can allow the user to visualize the hollow eye penetration member as the eye penetration member is being inserted into the proper insertion site of the patient's eye. FIGS. 5B and 6B illustrate the configuration of the device when tamponade pad 42 has been positioned against conjunctiva 20 of the patient's eye 16. Plunger 46 can then be depressed to urge therapeutic agent 48 from therapeutic agent reservoir 48 into a treatment site of the patient's eye 16 as depicted in FIGS. 5C and 6C. After therapeutic agent has been released, pressure can be applied to tamponade pad 42 by depressing outer housing 38, which translates to compressive force applied to conjunctiva 20 of the patient's eye 16. The eye penetration member can be manually withdrawn into outer housing 28 as illustrated in FIGS. 5D and 6D by retracting inner housing 40 proximally and pressure can be applied (or continue to be applied) to tamponade pad 42 by depressing outer housing 38 to apply (or continue to apply) compressive forces to conjunctiva 20 of patient's eye 16. FIG. 5E illustrates the configuration of the device after completion of the procedure.

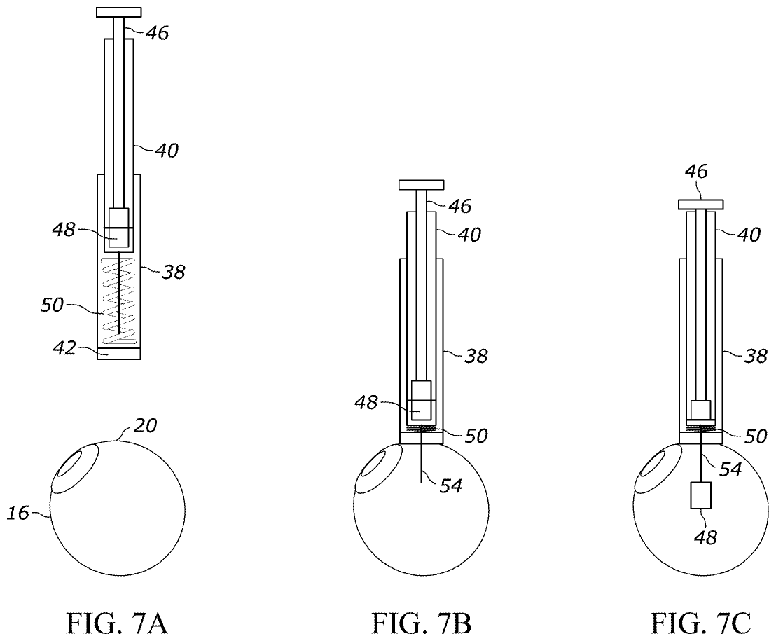

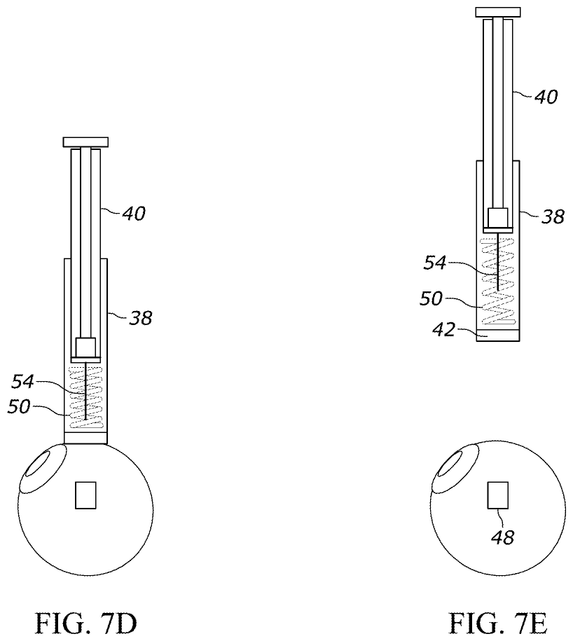

FIG. 4 depicts an embodiment of the intravitreal injection device of FIG. 3 with a self-retracting hollow eye penetration member. In particular, spring 50 can be located in outer housing 38 adjacent to hollow eye penetration member 54 and proximal to tamponade pad 42. In a compressed state of spring 50, hollow eye penetration member 14 extends distally beyond tamponade pad 42 and in an expanded state, hollow eye penetration member 14 extends proximal to tamponade pad 42. As stated above, a spring as depicted in FIG. 4 is only one example of how to implement a self-retracting hollow eye penetration member. Other components could also be utilized such that the hollow eye penetration self-retracts while pressure is preferably still applied to the tamponade pad. FIG. 7 illustrates the relative position of components of the intravitreal injection device depicted in FIG. 4 during different stages of deployment. FIG. 7A depicts hollow eye penetration member 54 disposed completely within outer housing 38 entirely proximal to tamponade pad 42 (similar to FIG. 5A) and with spring 50 in an expanded relaxed state. FIG. 7B illustrate the configuration of the device when tamponade pad 42 has been positioned against conjunctiva 20 of the patient's eye 16 and hollow eye penetration member 54 has been inserted into the eye 16. Plunger 46 can then be depressed to urge therapeutic agent 48 from therapeutic agent reservoir 44 into a treatment site of the patient's eye 16 as depicted in FIG. 7C. After a therapeutic agent(s) has been released, pressure can be applied to tamponade pad 42 by depressing outer housing 38, which translates to compressive force applied to conjunctiva 20 of the patient's eye 16. In FIG. 7D, spring 50 has been allowed to resume its relaxed, expanded state which biases the eye penetration member into outer housing 38 proximal to tamponade pad 42. Pressure can be applied (or continue to be applied) to tamponade pad 42 by depressing outer housing 38, to thereby apply compressive force to conjunctiva 20 of the patient's eye 16. FIG. 7E illustrates the configuration of the device after completion of the procedure.

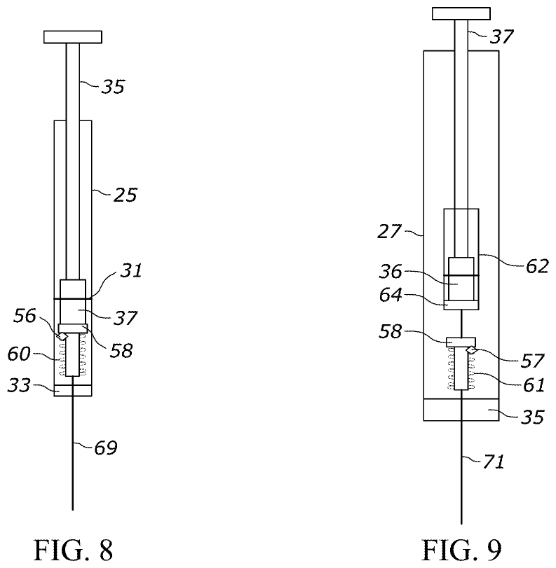

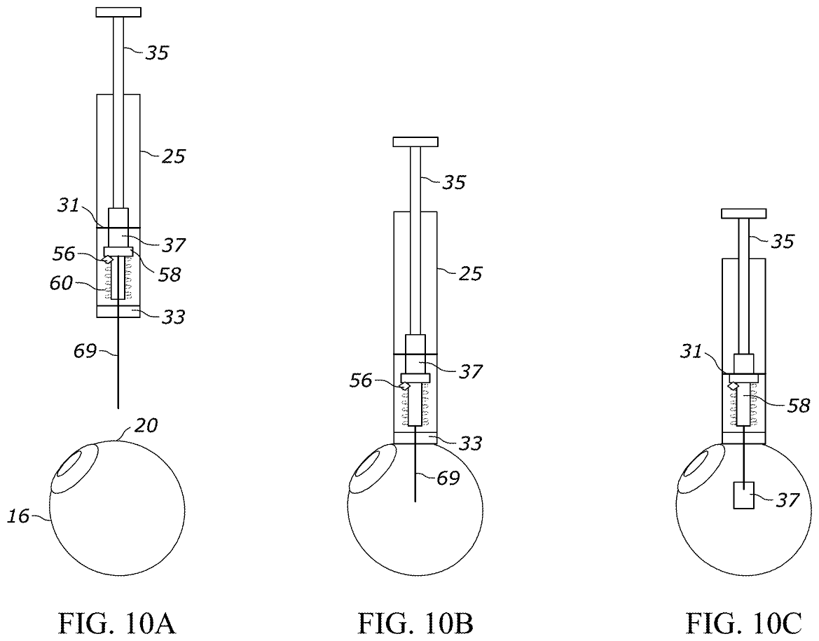

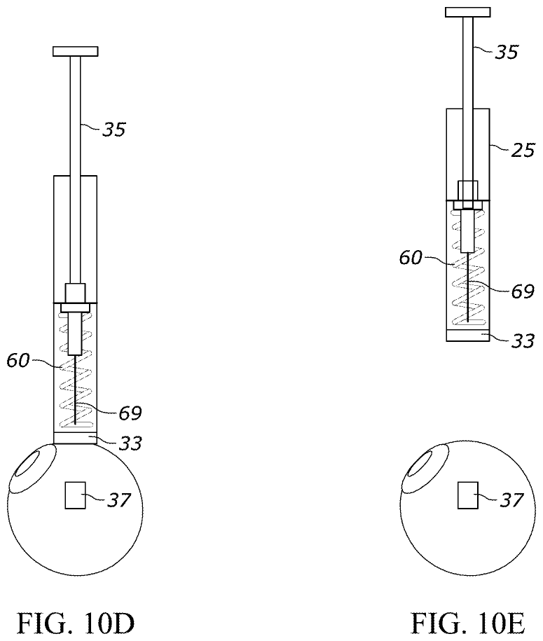

Referring to FIGS. 8 and 9, an intravitreal injection device can include a trigger or switch to assist in determining that delivery of the therapeutic agent is complete and to allow the hollow eye penetration member to have an initial exposed state. FIG. 8 illustrates a device with a therapeutic agent reservoir 31 and FIG. 9 illustrates a device with therapeutic agent cartridge 62. It should be noted that the term “therapeutic agent reservoir” as used herein may encompass the term “therapeutic agent cartridge.” Referring to FIG. 8, therapeutic agent reservoir 31 can be disposed within housing 25 and hollow eye penetration member 69 can be in fluid communication with therapeutic agent reservoir 31. The device can include an eye penetration member hub 58 connected to eye penetration member 69. Tamponade pad 33 can be located at the distal end of housing 25. Trigger or switch 56 can be located proximal to spring 60 and can be configured to retain spring 60 in a compressed state when trigger is in a latched position, which allows hollow eye penetration member to extend beyond tamponade pad 33 at the beginning of the procedure and is configured to allow spring 60 to assume a relaxed, expanded state when the trigger is an unlatched position such that the hollow eye penetration member is retracted into the housing proximal to the tamponade pad. FIG. 10 illustrates the relative position of components of the intravitreal injection device depicted in FIG. 8 during different stages of deployment. FIG. 10A illustrates the device with trigger 56 biased to a latched position to retain spring 60 in a compressed state. As such, hollow eye penetration member 69 extends distally beyond tamponade pad 33 and is therefore “exposed” and visible to the user. FIG. 10B illustrate the configuration of the device when tamponade pad 33 has been positioned against conjunctiva 20 of the patient's eye 16 and hollow eye penetration member 69 has been inserted into the eye 16. With reference to FIG. 10C, plunger 35 can be depressed to urge therapeutic agent 37 from therapeutic agent reservoir 31 into a treatment site of the patient's eye 16. Housing 25 can be depressed to apply pressure on tamponade pad to thereby apply compressive forces to conjunctiva 20 of eye 16 after delivery of the therapeutic agent (or additionally during insertion of the hollow eye penetration member and/or during delivery of the therapeutic agent). After delivery of the therapeutic agent, pressure on the plunger can be released and trigger 56 can be displaced from the latched position wherein spring 60 is free to assume a relaxed expanded position and hollow eye penetration member 69 is withdrawn into housing 25 under the bias of spring 60 as illustrated in FIG. 10D. Pressure (or continued pressure) can be applied to tamponade pad 33. FIG. 7E illustrates the configuration of the device after completion of the procedure.

Regarding FIG. 9, in order to release therapeutic agent 36, hollow eye penetration member 71 can have a sharpened exterior tip for penetrating the eye and a sharpened interior tip for penetrating septum 64 of therapeutic agent cartridge 62. Once cartridge 62 has been urged distally and septum 64 has been pierced, hollow eye penetration member 71 is in fluid communication with therapeutic agent cartridge 62. Housing 27, plunger 37, eye penetration member hub 58, trigger 57, and tamponade pad 35 can have similar structural features and functionalities as the same corresponding components illustrated in FIG. 8. FIGS. 8 and 9 illustrate one example of an intravitreal injection device that includes a trigger or switch to assist in determining that delivery of the therapeutic agent is complete and to allow the hollow eye penetration member to have an initial exposed state and contain a component such as a spring, for example, to bias the penetration member to a withdrawn state. Alternative components could also be used.

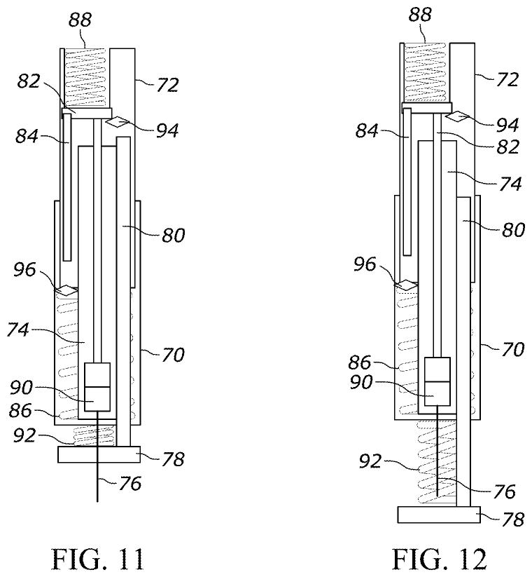

In certain aspects, an intravitreal injection device comprises a semi-automated therapeutic agent delivery system. FIG. 11 illustrates a device wherein the hollow eye penetrating member is initially “exposed” and FIG. 12 illustrates a device where the hollow eye penetrating member is initially “covered” in that it is proximal to the tamponade pad. In certain aspects, and with reference to FIG. 11, the device can comprise pad linker 80 operatively coupled to tamponade pad 78. Plunger 82 can be located proximal to therapeutic agent reservoir 74 (which, as stated above, in this instance and other embodiments also encompasses a therapeutic agent cartridge) and longitudinally displaceable within housing 70 to urge therapeutic agent 90 contained in therapeutic agent reservoir 74 into the patient's eye. Drug driving spring 88 located proximal to plunger 82 can bias plunger 82 towards therapeutic agent reservoir 74. Drug trigger 94 can be located between plunger 82 and pad linker 80. In FIG. 12, the distance between the drug trigger and the pad linker is greater than the distance between the drug trigger and the pad linker in FIG. 11. In certain aspects and further with respect to FIGS. 11-12, an intravitreal injection device can comprise outer housing 70 and proximal cap 72 slidably coupled with the outer housing 70. Therapeutic agent reservoir 74 can be slidably displaceable in outer housing 70 and proximal cap 72. Hollow eye penetration member 76 can be sized and dimensioned to be inserted into the vitreous humor of the patient's eye and in fluid communication with the therapeutic agent reservoir 74. Tamponade pad 78 can be configured to contact and apply pressure to a conjunctiva of a patient's eye and can be translationally coupled to the hollow eye penetration member 76. The translational coupling between tamponade pad 78 and the hollow eye penetration member 76 can allow the withdrawal of the hollow eye penetration member 76 while maintaining pressure between tamponade pad 78 and the conjunctiva. Pad linker 80 can axially extend in the outer housing 70 and the proximal cap 72 and can be operatively coupled to tamponade pad 78. Plunger 82 can slidably displaceable within the therapeutic agent reservoir. Plunger linker 84 can axially extend in outer housing 70 and proximal cap 72 and can be operatively coupled to a proximal end of plunger 82. Withdrawal spring 86 can be located in outer housing 70 adjacent to hollow eye penetration member 76 and proximal to tamponade pad 78. Drug driving spring 88 can be disposed in proximal cap 72 proximal to plunger 82 to bias plunger 82 towards therapeutic agent reservoir 74 to urge a therapeutic agent 90 in the therapeutic agent reservoir 74 into the patient's eye. A pad compression spring 92 can be located proximal to tamponade pad 78 to bias tamponade pad 78 toward the patient's eye. Drug delivery trigger 94 can be disposed in outer housing 70 between proximal end of plunger 82 and pad linker 80. Drug delivery trigger 94 can be configured to retain plunger 82 in a non-actuated state when the drug delivery trigger is in a latched position and to allow plunger 82 assume an actuated state when the drug delivery trigger is in an unlatched position. Withdrawal trigger 96 can be disposed in outer housing 70 between plunger linker 84 and withdrawal spring 86. Withdrawal trigger 96 can be configured to retain withdrawal spring 86 in a compressed state when the withdrawal trigger is in a latched position and can allow withdrawal spring to assume a relaxed expanded state when the withdrawal trigger is in an un-latched position. Such a device is exemplary and alternative components could be utilized for a semi-automated therapeutic agent delivery system.

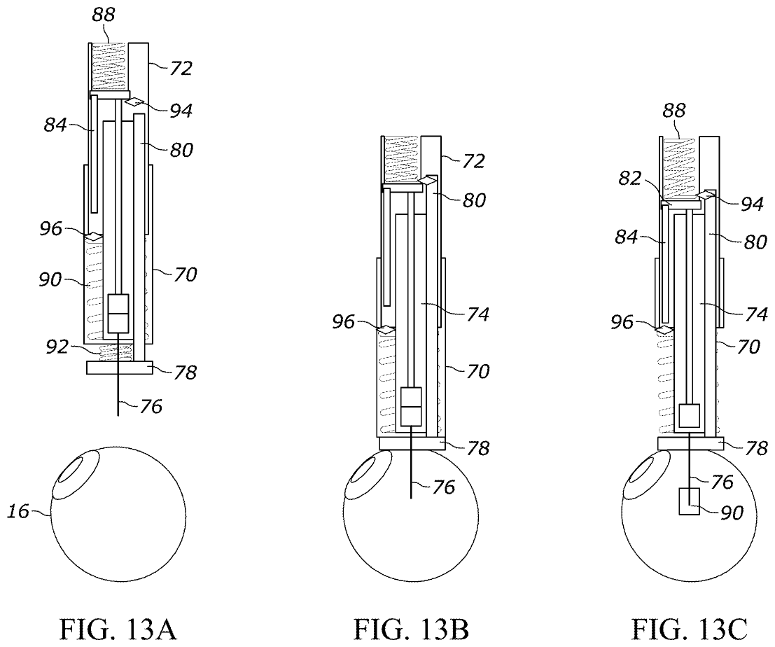

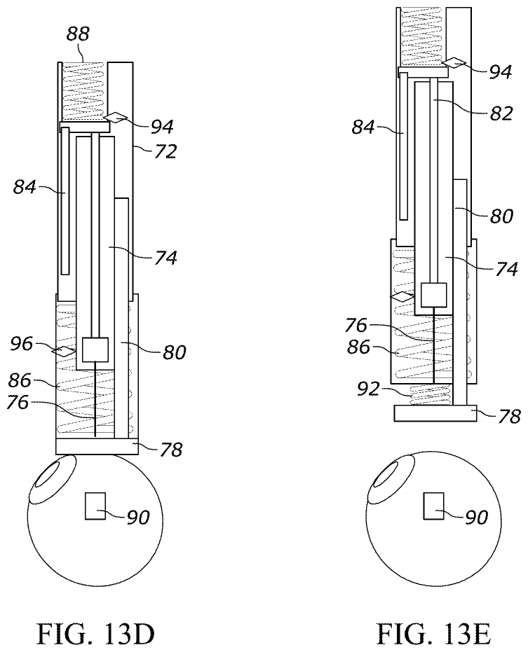

FIGS. 13A-13E illustrate the configuration of components of the device of FIG. 11 during different stages of deployment. FIG. 13A illustrates the configuration of the device at the beginning of the procedure where hollow eye penetration member 76 extends beyond tamponade pad 78 is therefore “exposed.” FIGS. 13B and 13C illustrate the configuration the device after the user has urged the hollow eye penetration member into the patient's eye and has applied pressure to tamponade pad 78. Such a step applies pressure to pad compression spring 92 and pad linker 80 contacts drug delivery trigger 94, which un-latches and releases the plunger 82 from an un-actuated state to an actuated state to urge therapeutic agent 90 into the patient's eye. When therapeutic agent has been fully delivered into the patient's eye, plunger linker 84 contacts withdrawal trigger 96 causing it to un-latch, which allows withdrawal spring 92 to assume an expanded state which biases hollow eye penetration member 76 into housing 70 as illustrated in FIG. 13D. Pressure can be applied (or continue to be applied) to tamponade pad 78 to apply (or continue to apply) compressive forces to the patient's eye. FIG. 13E illustrated the configuration of the components after completion of the procedure.

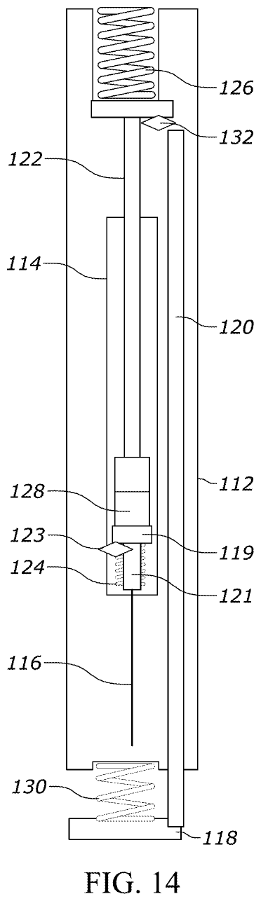

FIG. 14 is an example of an intravitreal injection device with a semi-automated hollow eye penetration member delivery system. The intravitreal injection device can comprise pad linker 120 operatively coupled to tamponade pad 118. Penetration member driving spring 126 can be located proximal to hollow eye penetration member 116 to bias the hollow eye penetration member into the patient's eye. Penetration member trigger 132 can be located between hollow eye penetration member 116 and pad linker 120. In certain aspects and further with reference to FIG. 14, an intravitreal injection device can comprise housing 112 and therapeutic agent reservoir 114 slidably displaceable in housing 112. Hollow eye penetration member 116 can be sized and dimensioned to be inserted into the vitreous humor of the patient's eye and can be in fluid communication with therapeutic agent reservoir 114. Hollow eye penetration member hub 121 can be operatively coupled to a proximal end of hollow penetration member 116. Tamponade pad 118 can be configured to contact and apply pressure to a conjunctiva of a patient's eye and can be translationally coupled to hollow eye penetration member 116. The translational coupling between the tamponade pad 118 and the hollow eye penetration member 116 can allow the withdrawal of hollow eye penetration member 116 while maintaining pressure between tamponade pad 118 and the conjunctiva. Pad linker 120 can axially extend in housing 112 and can be operatively coupled to tamponade pad 118. Plunger 122 can be slidably displaceable within the therapeutic agent reservoir 114. Withdrawal spring 124 can be located in housing 112 adjacent to hollow eye penetration member 116 and proximal to the tamponade pad 118. Withdrawal spring 124 can have a compressed state in which hollow eye penetration member 116 extends distally beyond tamponade pad 118 and an expanded state in which hollow eye penetration member 116 extends proximal to tamponade pad 118. In particular, withdrawal trigger 121 can be configured to retain withdrawal spring 124 in a compressed state when the withdrawal trigger is in a latched position and can allow withdrawal spring to assume a relaxed expanded state when the withdrawal trigger is in an un-latched position. Drug driving spring 126 can be disposed in housing 112 proximal to plunger 112 to bias plunger 112 towards therapeutic agent reservoir 114 to urge therapeutic agent 128 in therapeutic agent reservoir 114 into the patient's eye. Pad compression spring 130 can be located proximal to tamponade pad 118 to bias tamponade pad 118 toward the patient's eye. Drug delivery trigger 132 can be disposed in housing 112 between the proximal end of plunger 112 and pad linker 120. Withdrawal trigger 134 can be disposed in housing 112 adjacent a proximal portion 119 of hollow eye penetration member hub 121. Housing 112 can be depressed to apply pressure to tamponade pad 118 which, in turn, urges hollow eye penetration member 116 into the eye. Therefore, once pressure has been applied to the tamponade pad, the hollow eye penetration member is inserted into the eye. As such, insertion of the hollow eye penetration member into the eye is performed by the device when appropriate pressure has been applied to the tamponade pad as opposed to the user directly inserting the hollow eye penetration member into the patient. Such a device is exemplary and alternative components could be utilized for a semi-automated hollow eye penetration member delivery system.

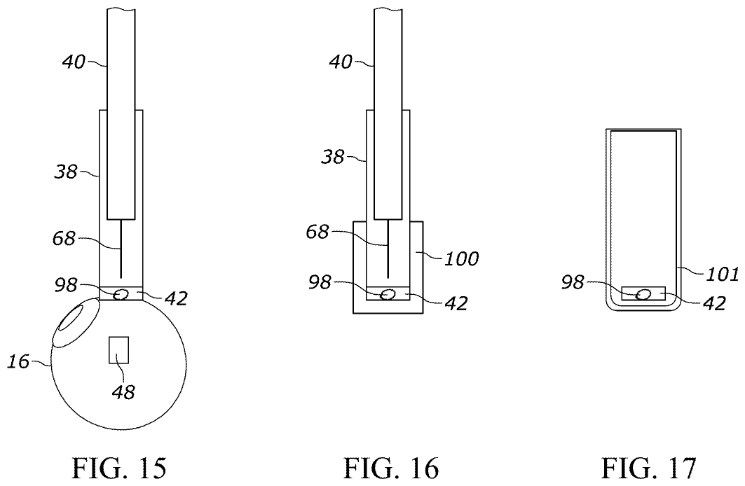

Referring to FIG. 15-17, tamponade pad 42 can comprises an absorptive material to absorb a vitreous gel or fluid sample 98 that has leaked out of the eye after, for example, therapeutic agent 48 has been delivered into eye 16 and hollow eye penetration member 68 has been retracted into housing 38. For example, the tamponade pad can comprise a polymer having a low surface energy and having pores sized to allow adsorption of a vitreous liquid or gel and minimize uptake of a vitreous strand such that an attachment of a vitreous can be broken when the tamponade pad is retracted (as it is advantageous that no attachment or minimal attachment of vitreous humor remains between the pad and the eye after pressure is relieved). Referring to FIG. 16, cover 100 can protect sample 98 until it is transferred, for example, to container 101 to be subsequently processed for analysis.

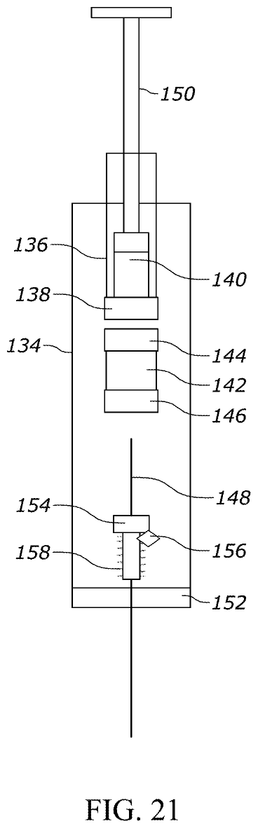

There are other ways in which an intravitreal injection device can be used to both delivery a therapeutic agent into the eye and to collect a sample from the eye such as a sample of vitreous fluid or gel. FIG. 21 illustrates an example of an intravitreal injection device that is both a drug delivery device and a vitreous gel or fluid aspiration device. The device can comprise housing 134 and therapeutic reservoir 136 (which is illustrated as a cartridge) slidably displaceable in housing 134. Therapeutic reservoir can contain a therapeutic agent 140 which can be urged out of the reservoir via plunger 150 which is slidably displaceable in housing 134. A pierceable septum 138 can be coupled to the distal end of therapeutic reservoir 136. Housing 134 can further comprise vacuum chamber 142 sealed by pierceable proximal septum 144 and pierceable distal septum 146. Hollow eye penetration member 148 can be partially located in housing 148 and extend through tamponade pad 152, which is located at the distal end of housing 134. Housing 134 can further include an eye penetration member hub 154 connected to eye penetration member 148 proximal to tamponade pad 152. Withdrawal trigger or switch 156 can be located proximal to withdrawal spring 158 and is configured to retain withdrawal spring 158 in a compressed state which allows hollow eye penetration member 148 to extend beyond tamponade pad 152 at the beginning of the procedure. The above-described drug delivery and aspiration device is exemplary and other devices can be used such as those disclosed in U.S. application Ser. No. 17/319,742, filed on May 13, 2021 and U.S. Application Ser. No. 17/750,563 filed on May 23, 2022, both of which are incorporated by reference herein.

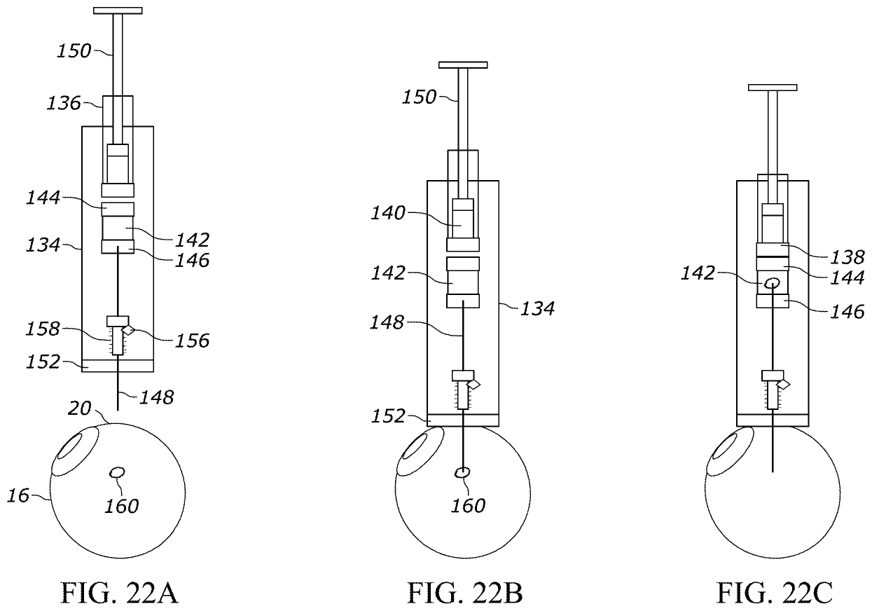

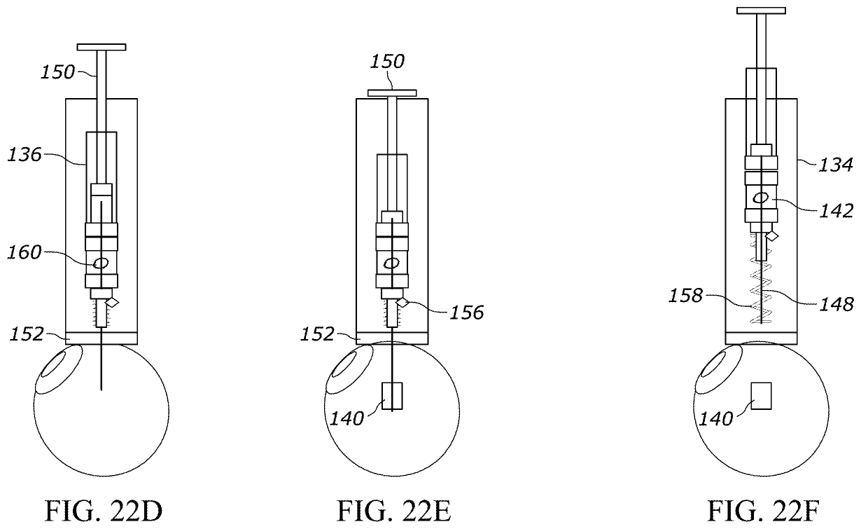

FIG. 22 illustrates the relative position of components of the intravitreal injection device depicted in FIG. 21 during different stages of deployment. FIG. 22A illustrates the device with trigger 156 biased to a latched position to retain spring 158 in a compressed state and where hollow eye penetration member 148 has partially pierced distal septum 146 such that hollow eye penetration member 148 is occluded such that vitreous fluid cannot pass out through the hollow eye penetration member 148, and hollow eye penetration member 148 is not yet in fluid communication with vacuum chamber 142. Hollow eye penetration member 148 extends distally beyond tamponade pad 152 and is therefore exposed and visible to the user. FIG. 22B illustrates the configuration of the device when tamponade pad 152 has been positioned against conjunctiva 20 of the patient's eye 16 and hollow eye penetration member 148 has been inserted into the eye 16 and is contact with a vitreous sample 160. FIG. 22C illustrates the configuration of the device after plunger 150 has translated a small distance forward such that the hollow eye penetration member 148 has completely penetrated distal septum 146 and is in fluid communication with the vacuum chamber. The differential pressure within eye 16 relative to the vacuum chamber 142 urges the sample 160 from eye 16 by hollow eye penetration member into vacuum chamber 142. In FIG. 22D, plunger 150 has been depressed such that hollow eye penetration member 148 is in fluid communication with therapeutic agent reservoir 136. Plunger 150 can be further depressed to urge therapeutic agent 140 from therapeutic agent reservoir 136 into a treatment site of the patient's eye 16 as depicted in FIG. 22E. Housing 134 can be depressed to apply pressure on tamponade pad to thereby apply compressive forces to conjunctiva 20 of eye 16 after delivery of the therapeutic agent (or additionally during insertion of the hollow eye penetration member and/or during delivery of the therapeutic agent). After delivery of the therapeutic agent, pressure on the plunger can be released and withdrawal trigger 156 can be displaced from the latched position wherein withdrawal spring 158 is free to assume a relaxed expanded position and hollow eye penetration member 148 is withdrawn into housing 134 under the bias of withdrawal spring 134 as illustrated in FIG. 22F.

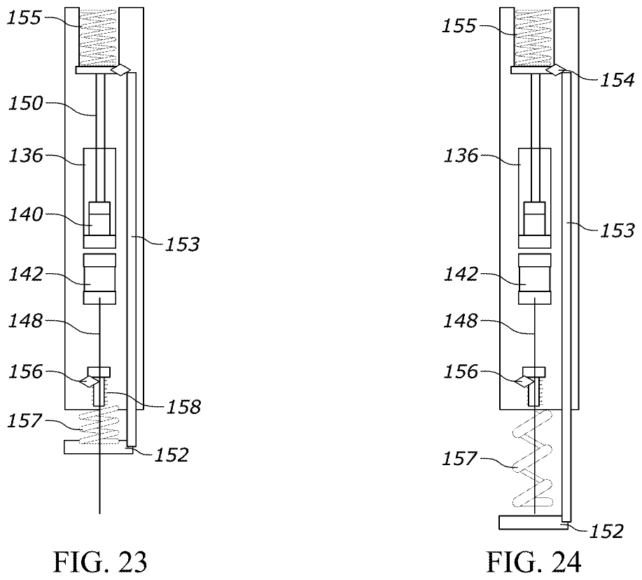

The intravitreal injection devices illustrated in FIGS. 23 and 24 are similar to the device illustrated in 21 but include components depicted in FIGS. 13 and 14. In particular, such devices include pad linker 153 operatively coupled to tamponade pad 152, drug driving spring 155 to bias plunger 150 towards the therapeutic agent reservoir 136, drug delivery trigger 154 located between plunger 150 and pad linker 153, withdrawal trigger 156, withdrawal spring 158, and pad compression spring 157. FIG. 23 illustrates the device with hollow eye penetration member 148 “exposed” and FIG. 24 illustrated hollow eye penetration member 148 “covered.”

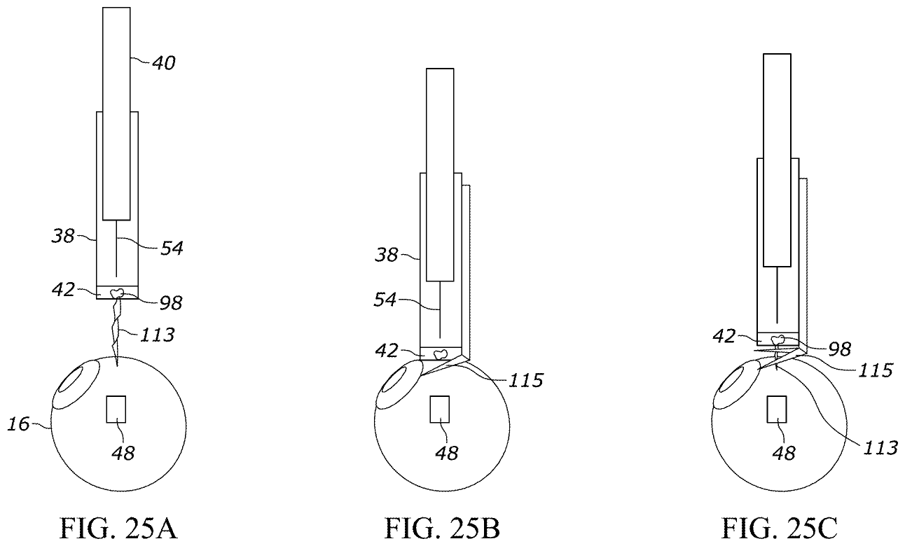

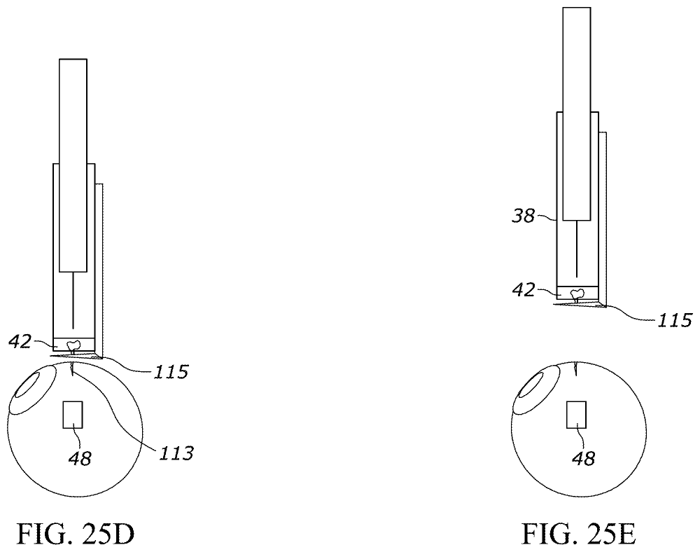

With reference to FIG. 25, an intravitreal injection device can comprise blades 115 disposed adjacent to tamponade pad 42 to sever vitreous strands 113 of the patient's eye 16 after pressure applied to the patient's conjunctiva by tamponade pad 42 is relieved. FIG. 25A depicts the configuration of a device without blades and illustrates the vitreous strands 113 that would otherwise be attached to sample 98. FIG. 25B depicts, for example, the configuration of a device with blades 116 when pressure is applied to tamponade pad 42 after injection of hollow eye penetration member 54 into the patient's eye 16 and delivery of therapeutic agent 48 to the treatment site. FIG. 25C depicts the configuration of the device when tamponade pad 42 has been removed from the surface of the eye and blades 115 have been actuated to sever vitreous strands 113. FIGS. 25D and 25E illustrate the configuration of the device after the vitreous strands 113 have been severed (FIG. 25D) and the procedure has been completed and the device withdrawn (FIG. 25E).

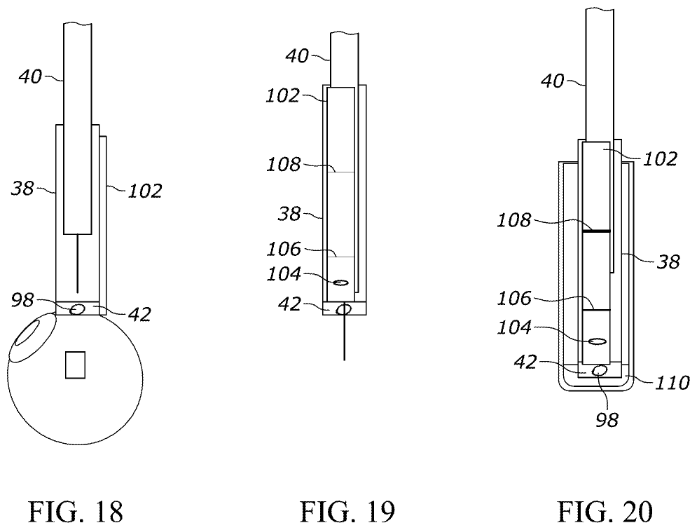

Referring to FIGS. 18-20, an intravitreal injection device can further comprise lateral flow assay 102 in fluid communication with tamponade pad 42. Lateral flow assay 102 can comprise a capillary substrate, a reporter antibody region 104 proximal to tamponade pad 42 comprising a reporter antibody that binds an epitope of an analyte in sample 98 of a vitreous fluid or gel from the vitreous humor. A test strip 106 can be located proximal to the reporter antibody region and comprise an antibody that binds an epitope of the analyte different from the epitope to which the reporter antibody binds. Control strip 108 can be located proximal to test strip 106 and can be configured to trap remaining reporter antibody unbound to the epitope in the analyte of sample 98. In particular, a sample and possibly additional carrier fluid from a solvent 110 may be drawn proximally by capillary action past an area of reporter antibody, a test strip, and a control strip. A highly visible band at the control strip confirms that a significant volume of dissolved antibody has crossed the control strip and therefore the test is considered valid. An observable band at the test strip indicates that some amount of measured analyte was present in the sample. The test strip may be read as observable versus non-observable or may be graded quantitatively in comparison to the test strip or other reference.

In intravitreal injection device may further include components to liquify the vitreous fluid such it may be more easily aspirated by the hollow eye penetration member. Ultrasound vibration, laser energy and other techniques may used to liquify the vitreous fluid. U.S. Provisional Application No. 63/396,426 filed on Aug. 9, 2022 entitled: “Proximal Ultrasonic Water Pressure Device for Ultrafine Gauge Vitrectomy in Combined Sampling and Drug Delivery Device,” which is incorporated by reference in its entirety describes an intravitreal drug delivery and sampling device that liquifies and removes vitreous gel from the eye using a needle probe inserted into the eye. The remainder of the vitreous remains largely unaltered and the liquefied sample can be collected by an extraction mechanism. Energy used to drive the liquefaction process can include acoustic vibration, a mechanical guillotine cutter, a mechanical screw cutter, electrical energy, optical energy, chemical action, and/or enzymatic action.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. An intravitreal injection device comprising:
a therapeutic agent reservoir;
a hollow eye penetration member sized and dimensioned to be inserted into the vitreous humor of a patient's eye and in fluid communication with the therapeutic agent reservoir; and
a tamponade pad configured to contact and apply pressure to a conjunctiva of the patient's eye and translationally coupled to the hollow eye penetration member, wherein the translational coupling between the tamponade pad and the hollow eye penetration member allows the withdrawal of the hollow eye penetration member while maintaining pressure between the tamponade pad and the conjunctiva.

2. The intravitreal injection device of claim 1, wherein the hollow eye penetration member is self-retracting.

3. The intravitreal injection device of claim 2, further comprising a spring located adjacent to the hollow eye penetration member and proximal to the tamponade pad, the spring having a compressed state in which the hollow eye penetration member extends distally beyond the tamponade pad and an expanded state in which the hollow eye penetration member extends proximal to the tamponade pad.

4. The intravitreal injection device of claim 1, further comprising a housing having a proximal end and a distal end, the therapeutic agent reservoir contained within the housing and the tamponade pad located at the distal end of the housing.

5. The intravitreal injection device of claim 4, further comprising a plunger located proximal to the therapeutic agent reservoir and longitudinally displaceable within the housing to urge a therapeutic agent contained in the therapeutic agent reservoir into the patient's eye.

6. The intravitreal injection device of claim 1, further comprising an outer housing and an inner housing slidably disposed in the outer housing.

7. The intravitreal injection device of claim 6, wherein:
the tamponade pad is located at the distal end of the outer housing; and
the therapeutic agent reservoir is located within the inner housing.

8. The intravitreal injection device of claim 7, further comprising a plunger located proximal to the therapeutic agent reservoir and longitudinally displaceable within the inner housing to urge a therapeutic agent contained in the therapeutic agent reservoir into the patient's eye.

9. The intravitreal injection device of claim 7, further comprising a spring located in the outer housing adjacent to the hollow eye penetration member and proximal to the tamponade pad, the spring having a compressed state in which the hollow eye penetration member extends distally beyond the tamponade pad and an expanded state in which the hollow eye penetration member extends proximal to the tamponade pad.

10. The intravitreal injection device of claim 1, wherein the tamponade pad is sized and dimensioned to serve as a guide to align the hollow eye penetration member at an appropriate location adjacent to the patient's eye.

11. The intravitreal injection device of claim 1, wherein a lateral distance from an edge of the tamponade pad to a location where the hollow eye penetration member penetrates the tamponade is between about 3.5 millimeters (mm) and about 4 mm.

12. The intravitreal injection device of claim 3, further comprising a trigger configured to retain the spring in its compressed state and configured to unlatch subsequent to release of a therapeutic agent from the therapeutic agent reservoir.

13. The intravitreal injection device of claim 3, further comprising a trigger configured to retain the spring in its compressed state, the trigger configured to unlatch subsequent to application of sufficient force upon the tamponade pad.

14. The intravitreal injection device of claim 1, further comprising a semi-automated therapeutic agent delivery system, wherein a therapeutic agent in the therapeutic agent reservoir is released through the hollow eye penetration member in response to displacement of the tamponade pad corresponding to complete insertion of the hollow eye penetration member.

15. The intravitreal injection device of claim 1, further comprising a semi-automated hollow eye penetration member deployment system wherein the hollow eye penetration member is extended distally in response to pressure applied to the tamponade pad.

16. The intravitreal injection device of claim 14, further comprising:
a pad linker operatively coupled to the tamponade pad;
a plunger located proximal to the therapeutic agent reservoir to urge a therapeutic agent contained in the therapeutic agent reservoir into the patient's eye;
a drug driving spring located proximal to the plunger to bias the plunger towards the therapeutic agent reservoir; and
a drug delivery trigger located between the plunger and the pad linker.

17. The intravitreal injection device of claim 15, further comprising:
a pad linker operatively coupled to the tamponade pad;
a penetration member driving spring located proximal to the hollow eye penetration member to bias the hollow eye penetration member into the patient's eye; and
a penetration member trigger located between the penetration member and the pad linker.

18. The intravitreal injection device of claim 1, further comprising a lateral flow assay in fluid communication with the tamponade pad.

19. The intravitreal injection device of claim 1, wherein the tamponade pad comprises an absorptive material to absorb a vitreous gel or fluid sample.

20. The intravitreal injection device of claim 1, wherein the tamponade pad comprises a polymer having a low surface energy and having pores sized to allow adsorption of a vitreous liquid or gel and minimize uptake of a vitreous strand such that an attachment of a vitreous strand can be broken when the tamponade pad is retracted.

21. The intravitreal injection device of claim 1, further comprising blades disposed adjacent to the tamponade pad configured to sever a vitreous strand of the patient's eye after pressure is relieved from the patient's conjunctiva.

22. The intravitreal injection device of claim 1 further comprising a vacuum chamber in fluid communication with the hollow eye penetration member configured to aspirate a sample of vitreous fluid or gel of the patient's eye through the hollow penetration member.
